(19) United States
(12) Patent Application Publication
(10) Pub. No.: US 2021/0012900 A1
(43) Pub. Date: Jan. 14, 2021

(54) METHOD FOR PRODUCING A ONE-NUMBER HEALTH SCORE FOR INDIVIDUALS TO MONITOR CHANGES TO THEIR WELLNESS AT A GLANCE

(71) Applicant: Darren Richard Smith, Orlando, FL (US)

(72) Inventor: Darren Richard Smith, Orlando, FL (US)

(21) Appl. No.: 16/509,998

(22) Filed: Jul. 12, 2019

(51) Int. Cl. G16H 50/30; G16H 10/60

(52) U.S. Cl. CPC G16H 50/30; G16H 10/60

(57) ABSTRACT

A comprehensive one-number health and wellness index rating is provided. The index score is adapted to facilitate individuals to track and improve their index rating and thus their health and wellness. The system embodied by the present invention aggregates, through a software application, a plurality of health metrics, including physician medical records, nutritional and biometric data, as well as health activities associated with time and date classifications; resulting in the health index rating.

### BACKGROUND OF THE INVENTION

The present invention relates to health monitoring methods and, more particularly, to a computer implemented method for producing a one-number health score to describe an individual's health and wellness status.

Most American adults are very aware, if not concerned about, their credit score. This one-number rating enables an overall sense of the associated individual's credit worthiness and general fiscal reliability, at a glance. As a result, people familiarize themselves with the key components that go into the credit score with the aim of maintaining or obtaining a good credit score.

There is no, unfortunately, a single number or rating that describes someone's health and wellness state taking into account behavior, activity, and biometrics. The current offerings are one dimensional and not complex enough to give a comprehensive view of the individual's health.

As can be seen, there is a need for a computer implemented method for producing a one-number health score to describe an individual's health and wellness status. The present invention aggregates different health and wellness metrics to produce a one number/score that describes the current health and wellness status for an individual. The present invention accounts for a plurality of measurable metrics and produces a single comprehensive complete rating for health and wellness. In short, the present invention embodies a health accountability index score accounting for nutrition, behavior, activity, and biometrics associated with the user's health and wellness status. Thereby, users can build a comprehensive and specific plan for improving their health, and at a glance measure their progress by monitoring their one-number health index.

### SUMMARY OF THE INVENTION

In one aspect of the present invention, computer-implemented system for producing a one-number health score for monitoring and improving an individual's health includes the following: providing a software application operatively associated with a mobile computing device, wherein the software application is configured to receive input associated with a plurality of health metrics; and representing the summation of the plurality of health metrics in a one-number health score.

In another aspect of the present invention, the computer-implemented system for producing a one-number health score for monitoring and improving an individual's health may embody the plurality of health metrics including a physician medical record; a nutritional data; a biometric data; and one or more health activities, each health activity associated with a time and date classification.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention; and

FIG. 2 is a detailed schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention for providing a comprehensive one-number health and wellness index rating for facilitating individuals to track and improve their index rating and thus their health and wellness. The system embodied by the present invention aggregates, through a software application, a plurality of health metrics, including physician medical records, nutritional and biometric data, as well as health activities associated with time and date classifications; resulting in the health index rating.

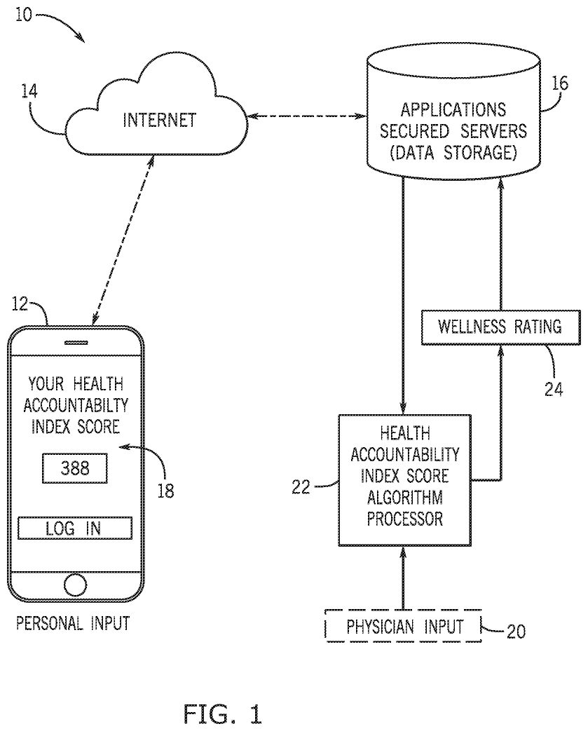

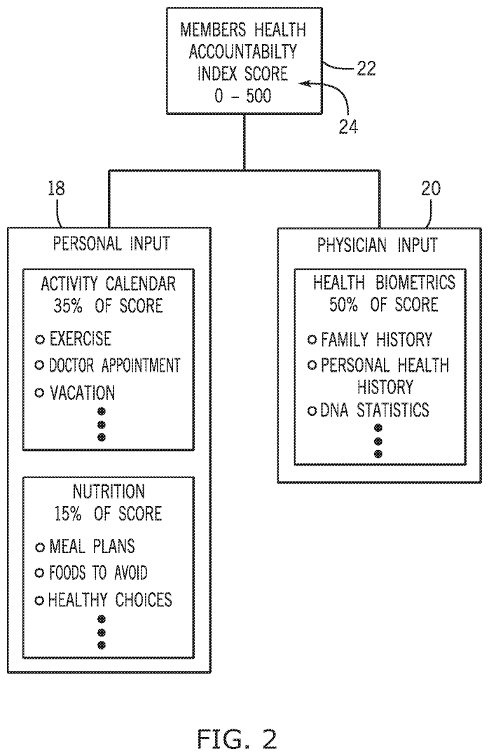

Referring to FIGS. 1 and 2, the present invention may include at least one computer 12 with a user interface. The computer 12 may include at least one processing unit coupled to a form of memory. The computer 12 may include, but not limited to, a microprocessor, a server, a desktop, laptop, and smart device, such as, a tablet and smart phone. The computer 12 includes a program product including a machine-readable program code for causing, when executed, the computer 12 to perform steps. The program product may include software which may either be loaded onto the computer 12 or accessed by the computer 12. The loaded software may include an application on a smart device. The software may be accessed by the computer using a web browser. The computer 12 may access the software via the web browser using the internet, extranet, intranet 14, one or more servers 16, internet cloud and the like.

The present invention embodies a health index system 10 including the following component steps: first, downloading the software application, tracking and inputting nutrition data 18; next associating predetermined activities and calendar of events; logging biometric data 18; updating the software application with the nutrition, biometric and activity-based data; receiving physician input 20, such as medical records including but not limited to the individual and family health history. The software application coupled to a health index algorithm 22 is adapted to produce the resulting health and wellness rating 24.

The health activities may be associated with exercise regimes, while the time and date classifications may be associated with the calendaring of said health activities at certain dates and times over a predetermine length of time.

A user may use the health rating 24 to gain an understanding of their current health and wellness state. It can then be used to influence their behavior in an effort to selectively improve their rating and thus improving their health and wellness.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A computer-implemented system for producing a one-number health score for monitoring and improving an individual's health, comprising:
providing a software application operatively associated with a mobile computing device, wherein the software application is configured to receive input associated with a plurality of health metrics; and
representing the summation of the plurality of health metrics in a one-number health score.

2. The computer-implemented system for producing a one-number health score for monitoring and improving an individual's health of claim 1, wherein the plurality of health metrics comprises:
a physician medical record;
a nutritional data;
a biometric data; and
one or more health activities, each health activity associated with a time and date classification.
